(19) United States
(12) Patent Application Publication
(10) Pub. No.: US 2013/0296562 A1
(43) Pub. Date: Nov. 7, 2013

(54) STEREOSELECTIVE PROCESS FOR PREPARATION OF 1,3-OXATHIOLANE NUCLEOSIDES

(71) Applicants: Bhairab Nath Roy, Pune (IN); Girij Pal Singh, Pune (IN); Dhananjai Srivastava, Pune (IN); Umesh Parasharam Aher, Pune (IN); Sudhakar Uttam Patil, Pune (IN)

(72) Inventors: Bhairab Nath Roy, Pune (IN); Girij Pal Singh, Pune (IN); Dhananjai Srivastava, Pune (IN); Umesh Parasharam Aher, Pune (IN); Sudhakar Uttam Patil, Pune (IN)

(73) Assignee: LUPIN LIMITED, Mumbai, Maharashtra (IN)

(21) Appl. No.: 13/979,502

(22) PCT Filed: Jan. 10, 2012
(86) PCT No.: PCT/IB12/50117

§ 371 Date: Jul. 12, 2013

(30) Foreign Application Priority Data
Aug. 5, 2011 (IN) 1038/KOL/2011

(51) Int. Cl. C07D 411/04

(52) U.S. Cl. CPC C07D 411/04
USPC 544/317

(57) ABSTRACT

The present invention relates to a stereoselective glycosylation for the preparation of 1,3-oxathiolane nucleoside in high yield and high optical purity. The invention specifically relates to a process of the preparation of Lamivudine and Emtricitabine using zirconium (IV) chloride (ZrCl₄) as a catalyst in glycosylation.

### OBJECT OF THE INVENTION

The main objective of the present invention is to provide an improved stereoselective glycosylation for preparing 1,3-oxathiolane nucleosides such as Lamivudine and Emtricitabine in cis-(−)-configuration in high yield and high optical purity using zirconium (IV) chloride (ZrCl₄) as a catalyst.

### BACKGROUND OF THE INVENTION

1,3-oxathiolane nucleosides, their analogues and derivatives are important class of therapeutic agent. 1,3-oxahiolane nucleosides and stereoisomers thereof having the general formula (I)

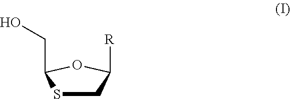

wherein R is substituted or unsubstituted purine or pyrimidine base or an analogues or derivatives thereof, have shown antiviral activity against retroviruses such as Human Immunodeficiency Virus (HIV), Hepatitis B Virus (HBV) and Human T-Lymphotrophic Virus (HTLV). Lamivudine and Emtricitabine are 1,3-oxathiolane nucleosides and presently available in the market as an antiretroviral agents.

Lamivudine is a cis-(−)-isomer and it is chemically known as (2R,cis)-4-amino-1-(2-hydroxymethyl-1,3-oxathiolan-5-yl)-(1H)-pyrimidine-2-one as represented by formula (Ia)

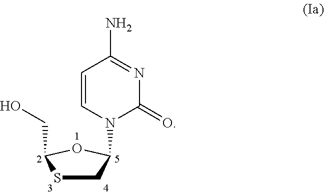

Emtricitabine is a cis-(−)-isomer and it is chemically known as (2R,cis)-4-amino-5-fluoro-1-(2-hydroxymethyl-1,3-oxathiolan-5-yl)-(1H)-pyrimidine-2-one as represented by formula (Ib)

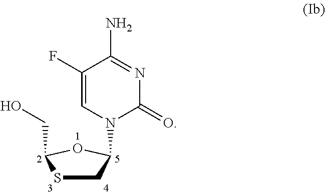

Lamivudine and Emtricitabine have two chiral centres and hence four stereoisomers are possible namely (2R,5S), (2S,5R), (2R,5R), (2S,5S) as shown below:

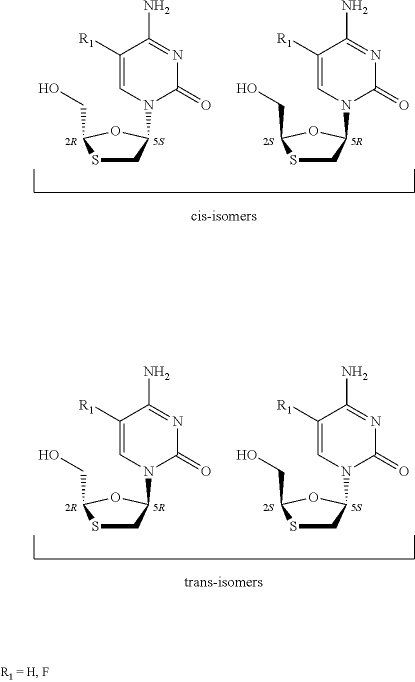

Cis (−) isomer, i.e. 2R,5S isomer is pharmaceutically more active and less cytotoxic.

The 1,3-oxathiolane nucleosides are prepared by condensation of 1,3-oxathiolane with pyrimidine or purine base by glycosylation. Glycosylation involves condensation of a sugar moiety with base such as purines, pyrimidines and derivatives thereof generally in presence of Lewis acid.

U.S. Pat. No. 5,047,407; U.S. Pat. No. 5,905,082 and J. Org. Chem., 1992, (57), 2217-2219 disclose condensation of oxathiolane of formula (II) or stereoisomer thereof,

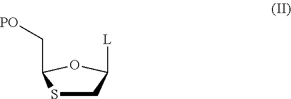

wherein P is protecting group and L is leaving group selected from OMe, OEt or OAc, with silyl and/or acetyl protected pyrimidine or purine base in the presence of Lewis acid such as trimethylsilyl triflate (TMSTf) or stannic chloride. The condensed product is finally deprotected to obtain desired oxathiolane nucleoside. However, the said documents do not provide process for synthesis of optically pure 1,3-oxathiolane nucleosides. Since, the process leads to mixture of isomers, one has to employ chromatography or enzymatic resolution to obtain Lamivudine.

The process disclosed in U.S. Pat. No. 5,047,407; U.S. Pat. No. 5,204,466 and J. Org. Chem., 1992 suffer from one of the following disadvantages.
The glycosylation reaction as provided in U.S. Pat. No. 5,047,407 is slow and takes around three days for completion. The yield of isolated product is low as the process leads to formation of all the four isomers in almost at an equal proportion.
The process provided in U.S. Pat. No. 5,204,466 and J. Org. Chem., 1992 employ use of stannic chloride (SnCl₄) as a Lewis acid for condensation of oxathiolane intermediate (II) with silylated cytosine or fluorocytosine. Besides the difficulty in handling the highly air and moisture sensitive, corrosive and fuming reagent, use of SnCl₄ in nucleoside synthesis are known to pose problems during work-up due to formation of emulsion that is difficult to break during extractions.
U.S. Pat. No. 5,204,466 reports reaction of 2-acetoxymethyl-5-acetoxy-1,3-oxathiolane with silylated cytosine or fluorocytosine using 3 mole equivalent of SnCl₄. The resulting product has been separated by flash chromatography rendering the process not user friendly.

U.S. Pat. No. 6,903,224 disclose the preparation of Lamivudine and Emtricitabine as a racemic mixture of both the geometric isomers. The process comprises reacting silylated cytosine or fluorocytosine with 2-benzyloxymethyl-5-ethoxy-1,3-oxathiolane in the presence of trimethylsilyl triflate (TMSTf) for 3 days under reflux to yield glycosylated product, which was isolated after chromatography as a mixture of cis and trans isomers (1:1). Thereafter the cis or trans isomer was deprotected in basic medium (methanolic NH₃) to yield racemic Lamivudine or Emtricitabine. As evident this is not the industrially desirable alternative where the glycosylation reaction is carried out for 3 days at reflux temperature. Further the reaction results in isomeric mixture (1:1) and the reaction product requires extra acylation step for isomer separation, which involves repeated chromatography and as a result of all these gives a very low overall yield of the racemic product.

U.S. Pat. No. 5,663,320 and U.S. Pat. No. 5,696,254 disclose a stereo-controlled synthesis of the desired cis-nucleoside analogue starting from optically pure intermediate. As per the process disclosed, the glycosylation is carried out in the presence of a silylated Lewis acid R₅R₆R₇XSi, wherein X is halogen and R₅, R₆, R₇ are selected from H, C₁₋₂₀ alkyl, e.g. trimethylsilyl iodide. There are number of disadvantages associated with the use of silylated Lewis acid as they are highly reactive, moisture sensitive and unstable compounds. The Lewis acid of choice, trimethylsilyl iodide (TMSI) is expensive and have significant toxicity.

Moreover as reported in Tet. Lett., 2005, (46), 8535-8538, the process involving trimethylsilyl iodide (TMSI) proves to be inefficient for preparing Lamivudine due to requirement of repetitive crystallization of the intermediate to obtain desired optical purity and hence leading to low yield.

U.S. Pat. No. 6,831,174 and U.S. Pat. No. 6,175,008 disclose a process to prepare racemic 1,3-oxathiolane nucleosides wherein the condensation of silylated pyrimidine bases such as uracil, cytosine derivatives with 1,3-oxathiolane moiety was carried out in the presence of Lewis acid such as TiCl₄, zinc chloride (ZnCl₂), TMSI, TMSTf, SnCl₄ or a mixture of ZnCl₂ and TiCl₄. Glycosylation using all these Lewis acids has resulted in the formation of mixture of cis and trans isomers. Further an additional step of acylation was required to separate the cis and trans isomers. The yield of mixture or individual isomer varies between 31 to 60% w/w. The process was further exemplified by glycosylation of 2-benzyloxymethyl-5-ethoxy-carbonyloxy-1,3-oxathiolane with silylated cytosine in presence of 0.3 mole of TiCl₄. The said reaction takes 3 hrs for completion and yields around 60% w/w of the glycosylated product with cis:trans ratio of 1:1.6. Since the process does not lead to chirally pure product, would not be commercially viable.

U.S. Pat. No. 6,939,965 discloses the glycosylation of silylated cytosine or 5-fluorocytosine with oxathiolane having a protected hydroxymethyl group at second position of the oxathiolane ring. The glycosylation is carried out using 1 mole of Lewis acid TiCl₃(OⁱPr) to give the desired (2R,5S) isomer in excess but required further purification by fractional crystallization to achieve pharmaceutically acceptable enantiopurity.

WO 2009/069011 discloses the process of preparation of compound of formula (I) from the compound of formula (III)

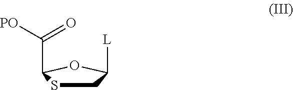

wherein L is the Leaving group preferably selected from acyl. The chiral auxiliary P of the compound of formula (III) is preferably L-menthyl group. The compound of formula (III) is reacted with pyrimidine base such as cytosine or fluorocytosine, wherein the amino or hydroxyl or both the groups of said bases are optionally protected with acetyl and/or silyl protecting groups. The glycosylation is carried out in the presence of a Lewis acid with the proviso that the Lewis acid does not contain any silyl groups. The Lewis acid is preferably stannic chloride (SnCl₄) or titanium tetrachloride (TiCl₄). The Lewis acid is used in about 0.5 to about 1.5 molar equivalents, preferably about 0.8 to 1.1 molar equivalents to the quantity of the compound of formula (III). The reaction is carried out in an organic solvent at a temperature of about 50° C. for about 10 min to about 100 hr. As a matter of fact use of stannic chloride (SnCl₄) or titanium tetrachloride (TiCl₄) for glycosylation is already disclosed in various documents as cited hereinbefore viz. U.S. Pat. No. 5,204,466; J. Org. Chem., 1992; U.S. Pat. No. 6,831,174 and U.S. Pat. No. 6,175,008. The application reports condensation of 1-menthyl-5-acetoxy-1,3-oxathiolane-2-carboxylate with N-acetyl silylated cytosine using 0.58 mole of SnCl₄, however, the process leads only to 11% (w/w) of yield, hence, would not be acceptable for scale-up.

WO 2010/082128 discloses the glycosylation of 1-menthyl-5-acetoxy-1,3-oxathiolane-2-carboxylate with N-acetyl silylated cytosine or fluorocytosine using 0.4 mole of trityl perchlorate at reflux for 12 hr to give 79:21 ratio of cis-(−):trans-(−) glycosylated product and hence requires further purification, which would further lead to loss in the yield.

Thus it is evident from the prior art that neither any Lewis acid catalyst used in glycosylation to obtain 1,3-oxathiolane nucleoside gave quantitative yield nor stereoselectivity but required further purification thus creating difficulty in product separation.

Therefore there is a need for an inventive, economically attractive, efficient and stereoselective synthesis of cis nucleoside analogues such as Lamivudine or Emtricitabine using a catalyst which is user friendly, eco-friendly, cost effective and the one giving high chemical yield with high optical purity.

Hence investigations have been directed towards the search of an appropriate catalyst including hitherto non-explored Lewis acid for the said glycosylation which not only gives high chemical yield but also high stereospecificity. Surprisingly use of tetravalent zirconium salt such as zirconium chloride satisfies most of the requirements.

### SUMMARY OF INVENTION

The present invention provides the process that comprises a step of glycosylating optionally silylated and/or acylated cytosine or fluorocytosine with an intermediate of formula (V)

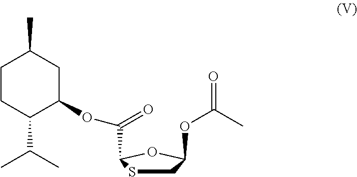

using zirconium chloride (ZrCl₄) as a catalyst at a temperature ranging between 20-40° C., preferably between 25-30° C., to obtain a compound of formula (VI) with cis-(−)-configuration stereoselectively almost in quantitative yield i.e. there is complete inversion of configuration at position 5 of 1,3-oxathiolane ring,

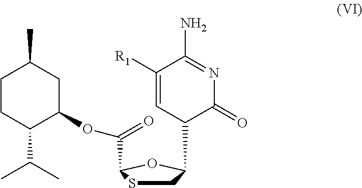

wherein R₁═H, F
which upon reduction with metal hydride gives Lamivudine of formula (Ia) or Emtricitabine of Formula (Ib)

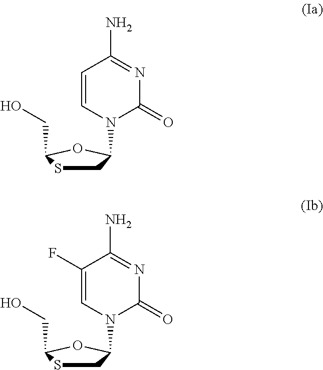

The process of the present invention also provides Lamivudine and Emtricitabine in high yield, high purity and high optical specificity.

The inventive merit of this invention lies in selection of a Lewis acid for glycosylation.

The glycosylation process of the present invention, specifically for Lamivudine and Emtricitabine, is shown below in scheme 1.

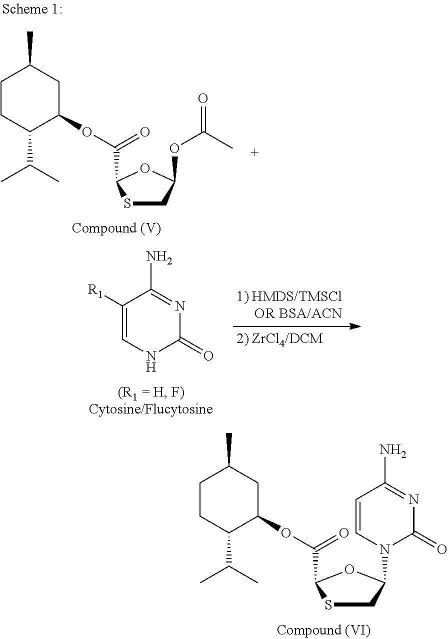

### DETAILED DESCRIPTION OF INVENTION

The present invention provides zirconium chloride (ZrCl₄) as a Lewis acid catalyst for glycosylation which gave quantitative yield with high optical purity of the 1,3-oxathiolane nucleoside.

It may not be out of place to summarize the difference between a Brønsted acid and Lewis acid. Ability of Lewis acid to complex with a lone pair of electrons is different for different acids. Hence one could expect different chemical prospective. Although in the literature number of Lewis acids like SnCl₄, TiCl₄, Cu-Triflate, TiCl₃(OⁱPr), TMSI has been used, they are not effective and none of them gave desirable results either in specificity or chemical yield. Each Lewis acid unlike Brønsted acid gives entirely different course of reaction [see Current Organic Chemistry, 2009, 13, 1-13; Titanium and Zirconium in Organic Synthesis, edited by Ilan Marck, Wiley-VCH Verlang GmbH and Co., KgaA, 2002, ISBN 3-527-30428-2]. This would be evident from the facts observed during our experiments using a number of Lewis acid such as SnCl₄, TiCl₄, Cu-Triflate, TMSI, Sc(SO₃CF₃)₃, BF₃.OEt₂ for the said glycosylation reaction, and surprisingly none of them gave any useful conversion. The difference between SnCl₄, TiCl₄, Cu-Triflate and TMSI could be evidence that there is a wide difference between Lewis acids. It would be evident from the Table No. 1 (provided in Example 3), which shows that ZrCl₄ gives the best specificity and highest chemical yield.

Contrary to Brønsted acid, where pKa is the essential factor for its efficacy, in Lewis acid the ability of complexing with a host molecule and extent of positive charge generation in the acceptor atom (e.g. complexing with carbonyl oxygen) determines the reaction feature of a Lewis acid, but it is not predictable and hence the course of reaction with a Lewis acid. Only innovative research work reveals the fact.

Hence finding an appropriate Lewis acid for a specific reaction itself is a complex phenomenon involving skill and requires extensive experimentation.

Hence investigations were carried with different Lewis acid catalysts for glycosylation. Lewis acids investigated for glycosylation are TiCl₄, SnCl₄, Cu-Triflate, and TMSI with more than 1 mole, but none of them gave more than 60% yield. Moreover these Lewis acids suffer from one or more disadvantages such as toxicity, cost and handling difficulty or hazards, non-feasibility towards various substituted or functionalized acylating reactants and the inconvenience of the procedure, especially extraction difficulty of the reaction mixture.

In the course of our investigation on the development of stereoselective glycosylation reaction for the efficient synthesis of Lamivudine it was discovered that the zirconium (IV) chloride (ZrCl₄), among various transition metal chlorides, is the reagent of choice for the glycosylation reaction. Zirconium (IV) chloride also known as zirconium chloride (ZrCl₄) is a high melting solid and used as a (weak) Lewis acid in organic synthesis. [see Current Organic Chemistry, 2009, 13, 1-13; Titanium and Zirconium in Organic Synthesis, edited by Ilan Marck, Wiley-VCH Verlang GmbH and Co., KgaA, 2002, ISBN 3-527-30428-2]. Unlike molecular TiCl₄, solid zirconium chloride (ZrCl₄) adopts a polymeric structure wherein each Zr is octahedrally coordinated. This difference in structure is responsible for the striking differences in their properties. TiCl₄ is distillable, but zirconium chloride (ZrCl₄) is a solid with a high melting point. Most zirconium compounds are relatively low toxic, easy to handle, of low cost and can tolerate small amount of moisture. Zirconium (IV) compounds have high coordinating ability that offers them as an adequate Lewis acid behaviour.

Zr⁴⁺ has a higher charge to size ratio (Z²/r=22.22 e²/m¹⁰) compared to most of the main group element and lighter and heavier transition metal ions such as Li⁺ (Z²/r=1.35 e²/m¹⁰), Bi³⁺ (Z²/r=8.82 e²/m¹⁰), In³⁺ (Z²/r=11.39 e²/m¹⁰), Sc³⁺ (Z²/r=12.33 e²/m¹⁰), Fe³⁺ (Z²/r=13.85 e²/m¹⁰), V³⁺ (Z²/r=14.06 e²/m¹⁰) and Al³⁺ (Z²/r=16.98 e²/m¹⁰) and is relatively softer hard acid (JOC, 2011, 76, 4753-4758). This inherent chemical feature of zirconium chloride (ZrCl₄) has led it to be a promising catalyst with strong acid behaviour, operational simplicity, and low toxicity. In addition its relatively high abundance and thus low cost offer attractive use in glycosylation.

The remarkable features of this catalytic glycosylation process are the mild reaction conditions, quantitative yields, short reaction times, high conversions, tolerability of various functional groups, clean reaction profiles, easy isolation of product and operational simplicity. Zirconium chloride (ZrCl₄) is an ideal Lewis acid since it is an efficient, stable, inexpensive, environmentally friendly and convenient catalyst.

Investigations were undertaken with different moles of zirconium chloride (ZrCl₄) per mole of substrate (Compound (V)), e.g. 0.1 mole, 0.3 mole and 0.5 mole for glycosylation reaction, but best yield was obtained with 0.5 mole which is less than the mole of other catalyst used for glycosylation. The mole wise experimental studies of zirconium chloride (ZrCl₄) used for glycosylation are comparatively shown in Table 2 (see Example 4).

According to the present invention the compound of formula (I)

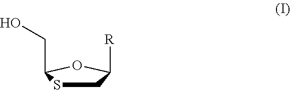

wherein R is substituted or unsubstituted purine or pyrimidine base or an analogues or derivatives thereof, is prepared by glycosylating silylated and/or acylated optionally substituted purine, pyrimidine base, analogues or derivatives thereof with an intermediate of formula (III)

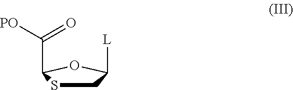

in presence of zirconium chloride (ZrCl₄) to obtain a compound of formula (IV)

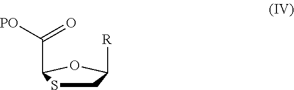

followed by reduction in presence of metal hydride.

The present invention provides a process for the preparation of Lamivudine and Emtricitabine, by using zirconium (IV) chloride (ZrCl₄) catalyst in glycosylation step.

The process comprises the step of glycosylating optionally silylated and/or acylated cytosine with intermediate of formula (V)

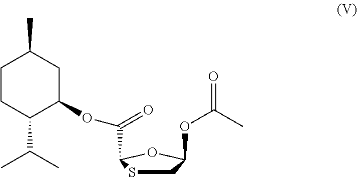

using zirconium chloride (ZrCl₄) as a Lewis acid catalyst to obtain a compound of formula (VI) with stereoselective cis configuration, i.e. there is complete inversion of configuration at position 5 of 1,3-oxathiolane ring,

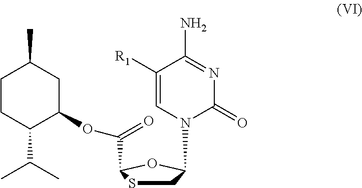

wherein R₁═H, F
which upon reduction with metal hydride such as NaBH₄, LiAlH₄, Li-triethyl borohydride or lithium-tri-sec-butyl borohydride to obtain Lamivudine or Emtricitabine.

There is complete inversion of configuration with quantitative yield in glycosylation when zirconium chloride (ZrCl₄) is used as a catalyst and obtained required cis-(−)-isomer. When the inventors conducted glycosylation of 2-benzoyloxymethyl-5-acetoxy-1,3-oxathiolane (with 40% cis and 60% trans configuration) with silylated cytosine or fluorocytosine there is obtained 60% cis and 40% trans glycosylated product (see example no. 8). This experimental results supports that there is 100% inversion in configuration.

The inventors have surprisingly found that 1,3-oxathiolane nucleoside such as Lamivudine or Emtricitabine can be prepared with high optical and chemical purity by using zirconium chloride in the condensation step. This invention also provides an efficient process to obtain 1,3-oxathiolane nucleosides with better yield. A chiral auxiliary may need to be used in executing the process but the use of optically pure intermediate and high temperature condition in the condensation step is not a necessary limitation of the present invention. The present process is suitable for preparing Lamivudine and Emtricitabine at an industrial scale.

The process of this invention may be used to prepare the compound of formula (Ia) and (Ib) and its pharmaceutically acceptable salt or ester thereof.

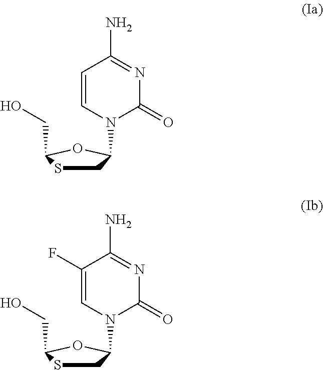

According to the present invention the hydroxyl group of the compound of formula (VII) is converted to an acyl group with acetic anhydride in an appropriate organic solvent such as dichloromethane, chloroform or pyridine to get a compound of formula (V) which upon glycosylation with optionally silylated cytosine or fluorocytosine, in the presence of zirconium chloride (ZrCl₄) which is a key feature of the present invention to obtain a compound of formula (VI). The ester group of compound of formula (VI) is selectively reduced with sodium borohydride (NaBH₄) in methanol to yield Lamivudine or Emtricitabine shown below in scheme 2.

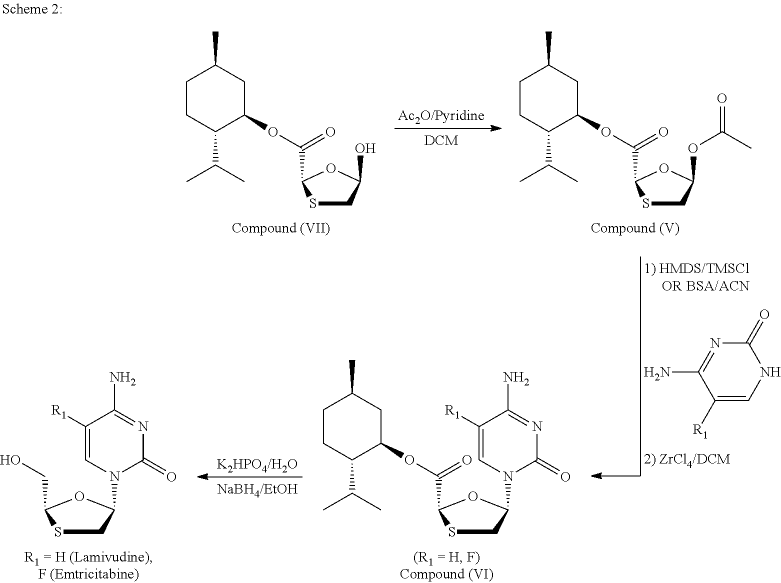

The present invention provides a process for the preparation of Lamivudine and Emtricitabine of formula (Ia) and (Ib) respectively,

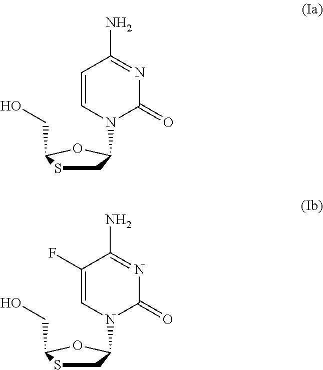

the process comprises of—
a) reacting a compound of formula (V)

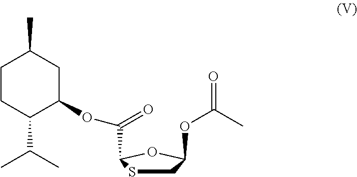

with optionally silylated and/or acylated cytosine or fluorocytosine wherein the amino or hydroxyl or both the group of said cytosine or fluorocytosine base are optionally protected with protecting group, in the presence of zirconium chloride (ZrCl₄) at a temperature ranging between 20 to 40° C., preferably between 25 to 30° C. to obtain a compound formula (VI);

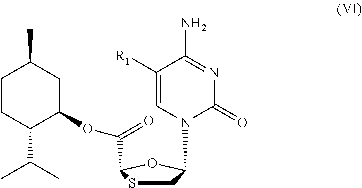

wherein R₁═H, F
b) reducing the compound of formula (VI) to obtain Lamivudine or Emtricitabine of formula (Ia) or (Ib);
c) isolating Lamivudine or Emtricitabine of formula (Ia) or (Ib) from the reaction mixture.

The present invention described the process of preparation of Lamivudine and

Emtricitabine using zirconium chloride (ZrCl₄) as a catalyst used for glycosylation. The complete process is described below.

Reaction of L-menthyl glyoxylate hydrate with 1,4-dithiane-2,5-dione in the presence of toluene and acetic acid under reflux temperature and removing water azeotropically gave L-menthyl-5R-hydroxy-1,3-oxathiolanes-2R-carboxylate, which upon acylation with acetic anhydride in dichloromethane (DCM) and pyridine to get L-menthyl-5R-acetoxy-1,3-oxathiolane-2R-carboxylate which upon glycosylation with optionally silylated and/or acetylated cytosine or fluorocytosine using zirconium chloride (ZrCl₄) as a catalyst (which is a key feature of the present invention) in dichloromethane to obtain glycosylated product which is having cis-(−)-configuration with chiral purity at least 97%. The glycosylated product upon reduction with sodium borohydride in ethanol in the presence of di-potassium hydrogen phosphate in water to get Lamivudine or Emtricitabine with a chiral purity at least 98%. Thus obtained Lamivudine or Emtricitabine was further purified by making its complex with (S)-1,1′-Bi-2-naphthol ((S)-BINOL) to obtain Lamivudine or Emtricitabine with high chiral purity (at least 99% ee). The complete reaction scheme is shown below in scheme 3.

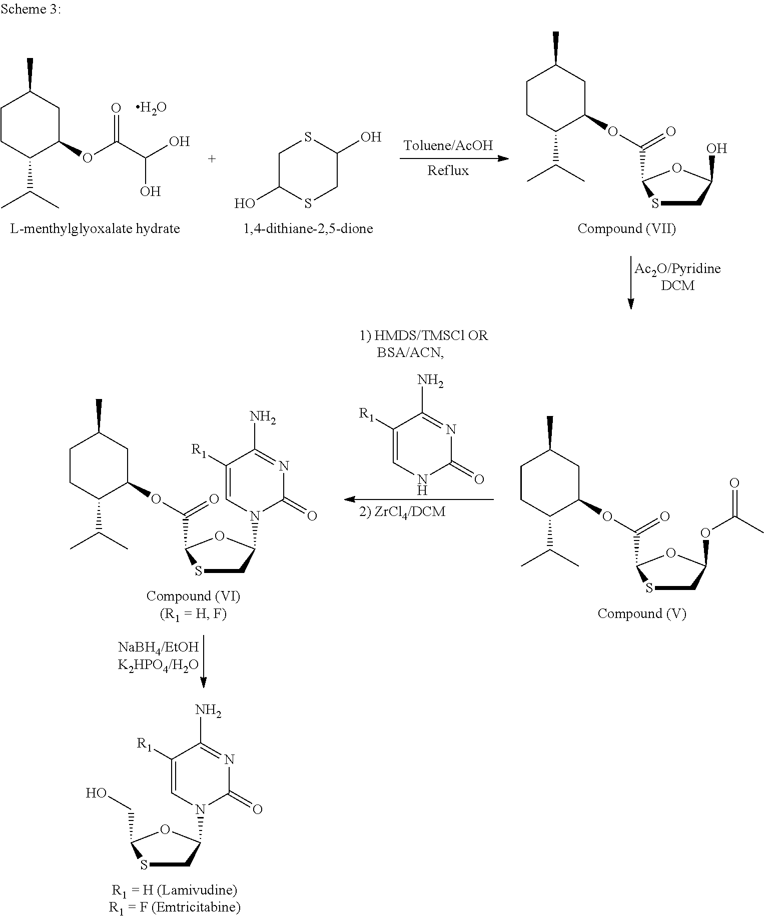

Lamivudine obtained according to the process of the present invention is having purity at least 98%, i.e. there is 98% ee Lamivudine and 2% cis-(+)-Lamivudine. The Lamivudine thus obtained was further enriched using S-(−)-BINOL which gave 99.25% ee Lamivudine having around 0.75% cis-(+) isomer of Lamivudine, which was further enriched by repeating complexation with S-BINOL to obtain 99.8% ee Lamivudine. (see Org. Process Res. Dev. 2009, 13, 450-455)

The chiral purity of Lamivudine and Emtricitabine obtained according to the process of present invention was at least 98%, preferably 99.25%, more preferably 99.8%.

Protection and deprotection wherever performed in the present invention adopts the general methods used in organic chemistry as described in Greene and Wuts (protective groups in Organic Synthesis, Wiley and sons, 1999) such as but are not limited to, tert-butoxycarbonyl (Boc), benzyloxycarbonyl (Cbz), benzyl(Bn), acetyl and trifluoroacetyl.

The invention is further described by the following examples which are not intended to limit the scope of invention in any way.

### Example 1

### Preparation of L-menthyl-5R-hydroxy-1,3-oxathiolanes-2R-carboxylate

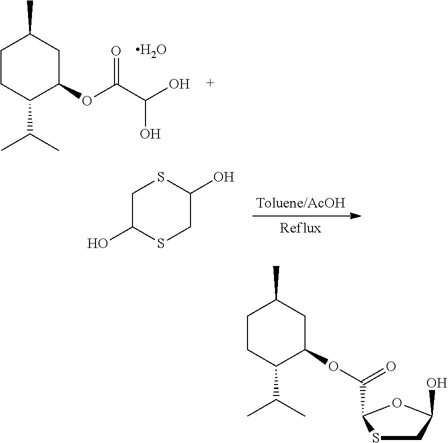

L-menthyl glyoxylate hydrate (prepared by reaction of L-menthol with glyoxalic acid as per process described in Synthetic Commun., 1990, 20, 2837-2847 by Fernadez F.) (100 gm, 0.434 mol, 1 molar equivalents), toluene (500 ml) and acetic acid (10 ml) were mixed under stirring and thus formed reaction mixture was heated up to 110-115° C., to remove water azeotropically. The reaction mixture was cooled up to 80° C. and solvent was distilled under vacuum up to the final volume becomes 300 ml. The reaction mixture was cooled to 50° C. and 1,4-Dithiane 2,5-Diol (33.1 gm, 0.217 mol, 2 molar equivalents) was added. The reaction mixture was refluxed (110-115° C.) and monitored the reaction by TLC. The mixture was cooled to 0-5° C. after completion of the reaction. 600 ml of 10% triethylamine in n-heptane was added to the reaction mixture drop wise over a period of an hour. The mixture was then maintained at 0-5° C. for an hour to form a solid. The isolated solid was filtered, washed with n-heptane and dried. Yield: 100 g. ¹H NMR (DMSO-d6+D₂O) δ (ppm): 0.70-0.91 (m, 10H); 0.95-1.05 (q, 2H), 1.35-1.46 (q, 2H), 1.62-1.64 (d, 2H), 1.84-1.90 (t, 2H), 2.86-2.88 (d, 1H), 3.12-3.16 (q, 1H), 4.59-4.64 (m, 1H), 5.55-5.60 (t, 1H), 5.86 (s, 1H), 7.03-7.04 (d, 1H); ¹³C NMR (DMSO-d6) δ (ppm): 21, 22.2, 23.3, 26, 31.2, 34, 37.8, 38, 40.1, 46.7, 75, 76.4, 102.4, 169.5; IR (KBr) (cm⁻¹): 3470, 2962, 29.34, 28.366, 1734, 1465, 1198, 1077, 1041, 902, 516; MS (EI) m/z=287.3 (M−1).

### Example 2

### Preparation of L-menthyl-5R-acetoxy-1,3-oxathiolane-2R-carboxylate

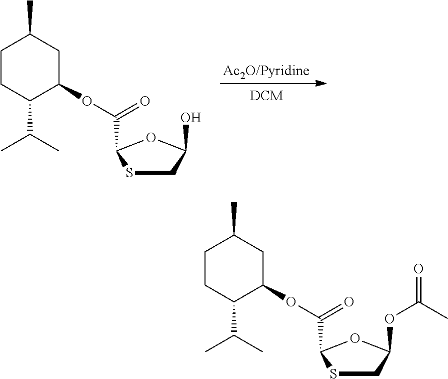

L-menthyl-5R-hydroxy-1,3-oxathiolanes-2R-carboxylate (100 g, 0.346 mol, 1 molar equivalents), acetic anhydride (200 ml, 2.119 mol, 6.1 molar equivalents) and dichloromethane (500 ml) at 25-30° C. were mixed under nitrogen atmosphere. The reaction mixture was cooled to 0-5° C. and pyridine (50 ml) was added drop wise under stirring. The reaction mixture was stirred for 2-3 hours while maintaining the same temperature. The reaction was monitored with TLC and after completion it was quenched with water at 5-10° C. The mixture was stirred, settled and the layers were separated. The organic layer was washed with dilute HCl and concentrated under vacuum. n-Heptane (500 ml) was added to the residue and then heated to become a clear solution at about 60-65° C. The solution was cooled gradually up to room temperature and further to 0-5° C. The isolated solid product was filtered, washed with pre-cooled (0-5° C.) n-heptane and dried under vacuum at 40-45° C. Yield: 80 g. ¹H NMR (CDCl₃) δ (ppm): 0.70-2.11 (m, 21H), 3.15-3.18 (m, 1H), 3.43-3.47 (m, 1H), 4.70-4.77 (m, 1H), 5.62 (s, 1H), 6.8 δ (m, 1H); ¹³C NMR (CDCl₃) δ (ppm): 16.1, 21, 21.9, 23.2, 26, 31.3, 34.1, 37.2, 40.6, 47, 76.1, 77, 79.9, 99.7, 168.6, 169.7; IR (KBr) (cm⁻¹): 3446, 2955, 2932, 2873, 1749, 1732, 1459, 1377, 1312, 1241, 1190, 1148, 1103, 1024, 950, 853, 733, 597; MS (EI) m/z=348 (M+NH₄); [α]_{D}²⁰=−62.09° (c=0.5%, CHCl₃); Melting Range: 101.7-102.2° C.

### Example 3

### Preparation of L-menthyl-5S-(4-amino-2-oxopyrimidin-1-(2H)-yl)-1,3-oxathiolane-2R-carboxylate using various Lewis acids

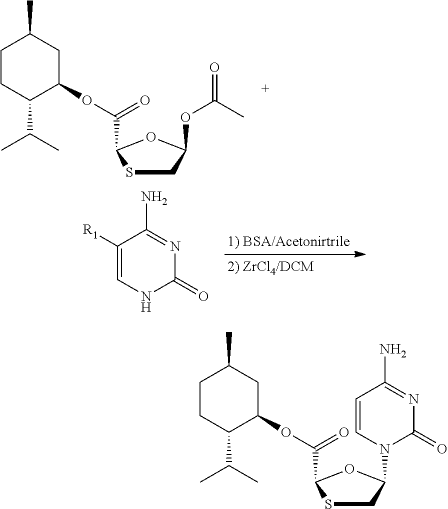

Cytosine (1.2 molar equivalents), N,O-bis-(trimethylsilyl)-acetamide (BSA) (2.7 molar equivalents) and acetonitrile (ACN) (5 volume) were mixed under nitrogen atmosphere at 25-30° C. to get clear solution. The solution was distilled out to remove excess BSA and acetonitrile completely under vacuum at 50-55° C. to get the residue of silylated cytosine into which fresh solvent was added (solution A). In a separate flask L-menthyl-5R-acetoxy-1,3-oxathiolane-2R-carboxylate (1.0 molar equivalents) and catalyst, as provided in table 1, in a solvent were stirred under nitrogen atmosphere (solution B). Solution A was mixed into solution B under stirring at 25-30° C. and continued the stirring at the same temperature for 12-18 hrs. The reaction was monitored by HPLC or thin layer chromatography. After completion of reaction, the reaction mixture was cooled to 10-20° C. into which the saturated bicarbonate solution (5 volume) was added under stirring. The layers were separated and the organic layer was concentrated under vacuum to get the residue. To the residue isopropyl acetate was added under stirring. The mixture was cooled to 5-10° C. and maintained for 2-3 hours. The isolated solid was filtered and washed with isopropyl acetate. The solid was dried under vacuum at 40-45° C. ¹H NMR (DMSO-d6) δ (ppm): 0.70-0.90 (m, 10H), 0.99-1.07, (m, 2H), 1.38-1.47 (m, 2H), 1.63-1.66 (d, 2H), 1.88-1.94 (t, 2H), 3.09-3.14 (m, 1H), 3.51-3.55 (m, 1H), 4.63-4.70 (m, 1H), 5.68 (s, 1H), 5.77-5.79 (d, 1H), 6.32-6.35 (t, 1H), 7.30-7.33 (d, 2H), 7.94-7.96 (d, 1H); ¹³C NMR (DMSO-d6) δ (ppm): 16.5, 20.9, 22.2, 23.2, 26.1, 31.2, 33.9, 35.6, 39.9, 46.8, 75.8, 77.7, 89.2, 94.7, 140.9, 155.1, 166.1, 169.6; IR (KBr) (cm⁻¹): 3358, 3177, 2975, 2869, 1753, 1634, 1488, 1368, 1288, 1180, 1087, 981, 786, 596; MS (EI) m/z=382.2 (M+1); [α]_{D}²⁵=−102.22° (c=0.5%, MeOH).

The comparative experimental condition for glycosylation and results for different catalyst are shown in Table 1.

TABLE 1
Experimental conditions and results for different catalyst

| | | Molar equivalent | | | | |
|---|---|---|---|---|---|---|
| Sr. | | with respect to | | Reaction | | % Ratio of |
| No. | Catalyst | compound (V) | Solvent | Time (Hr) | Yield (%) | cis-(−):Unknown |
| 1. | ZrCl₄ | 0.5 | DCM | 12 | 80 | 97.69:2.31 |
| 2. | TiCl₄ | 1.0 | DCM | 12 | * | — |
| 3. | SnCl4 | 1.0 | DCM | 12 | * | — |
| 4. | Cu-Triflate | 1.0 | DCM | 12 | * | — |
| 5. | TMSI | 1.5 | DCM | 18 | 60 | 99.85:0.15 |
| 6. | Sc(SO₃CF₃)₃ | 1.0 | DCM | 12 | * | — |
| 7. | BF3•OEt₂ | 1.0 | DCM | 12 | * | — |
| 8. | TiCl₄ | 0.5 | Acetonitrile | 12 | * | — |

* Product was not isolated as the conversion was less than 5%.

### Example 4

### Preparation of L-menthyl-5S-(4-amino-2-oxopyrimidin-1-(2H)-yl)-1,3-oxathiolane-2R-carboxylate using varying mole proportions of zirconium chloride

Cytosine (1.2 molar equivalents), N,O-bis-(trimethylsilyl)-acetamide (BSA) (2.7 molar equivalents) and acetonitrile (5 volumes) were mixed under nitrogen atmosphere at 25-30° C. to get a clear solution. The solution was distilled out under vacuum at 50-55° C. to remove excess BSA and acetonitrile completely to get the residue of silylated cytosine into which fresh solvent was added (solution A). In a separate flask L-menthyl-5R-acetoxy-1,3-oxathiolane-2R-carboxylate (1.0 molar equivalents), zirconium chloride (quantities as per table 2) and dichloromethane (DCM) (5 volumes) were mixed under nitrogen atmosphere (solution B). Solution A was added into solution B under stirring at 25-30° C. and continued the stirring at the same temperature for 6 hrs. Reaction was monitored by HPLC or thin layer chromatography. After the completion of reaction, the reaction mixture was cooled to 10-20° C. and saturated bicarbonate solution (5 volumes) was added under stirring. The layers were separated and the organic layer was concentrated under vacuum to get the residue. To the residue isopropyl acetate was added under stirring. The mixture was cooled to 5-10° C. and maintained for 2-3 hours. The isolated solid was filtered and washed with isopropyl acetate. The solid was dried under vacuum at 40-45° C.

The mole wise comparison of ZrCl₄ used for glycosylation and yield of glycosylated product are shown in Table 2.

TABLE 2
Experimental conditions and results for different
proportions of zirconium chloride catalyst.

| | ZrCl4 molar | | | |
|---|---|---|---|---|
| | equivalents | Reaction | | Ratio of |
| Sr. | with respect to | Time | Yield | cis-(−): |
| No. | compound (V) | (Hr) | (%) | Unknown |
| 1. | 0.1 | 6 | 30 | 98.53:1.47 |
| 2. | 0.3 | 6 | 45 | 97.50:2.50 |
| 3. | 0.5 | 6 | 80 | 98.15:1.85 |
| 4. | 1.0 | 12 | 78 | 98.45:1.55 |

### Example 5

### Preparation of L-menthyl-5S-(4-amino-2-oxopyrimidin-1-(2H)-yl)-1,3-oxathiolane-2R-carboxylate

Cytosine (20 g, 0.180 mol, 1.2 molar equivalents), N,O-bis-(trimethylsilyl)-acetamide (BSA) (100 ml, 0.408 mol, 2.7 molar equivalents) and acetonitrile (100 ml) were mixed under nitrogen atmosphere at 25-30° C. to get clear solution. The solution was distilled under vacuum at 50-55° C. to remove excess BSA and acetonitrile completely to get the residue of silylated cytosine into which the dichloromethane (100 ml) was added (solution A). In a separate flask L-menthyl-5R-acetoxy-1,3-oxathiolane-2R-carboxylate (50 g, 0.151 mol, 1.0 molar equivalents), zirconium tetrachloride (17.6 g, 0.075 mole, 0.5 molar equivalents) and dichloromethane (500 ml) were mixed under nitrogen atmosphere (solution B). Solution A was added into solution B under stirring at 25-30° C. and continued the stirring at the same temperature for 6 hrs. The reaction was monitored by HPLC or thin layer chromatography. After completion of reaction, the reaction mixture was cooled to 10-20° C. in which saturated bicarbonate solution (50 ml) was added under stirring. The layers were separated and the organic layer was concentrated under vacuum to get the residue. To the residue isopropyl acetate (5-10 ml) was added under stirring. The mixture was cooled to 5-10° C. and maintained for 2-3 hours. The isolated solid filtered and washed with isopropyl acetate. The solid was dried under vacuum at 40-45° C. Yield: 45 g. Chiral Purity: 97% and 3 undesired isomer.

### Example 6

### Preparation of L-menthyl-5S-(4-amino-2-oxopyrimidin-1-(2H)-yl)-1,3-oxathiolane-2R-carboxylate

Cytosine (20 g, 0.180 mol, 1.2 molar equivalents), N,O-bis-(trimethylsilyl)-acetamide (BSA) (100 ml, 0.408 mol, 2.7 molar equivalents) and acetonitrile (100 ml) were mixed under nitrogen atmosphere at 25-30° C. to get clear solution. The solution was distilled under vacuum at 50-55° C. to remove excess BSA and acetonitrile completely to get the residue of silylated cytosine into which the dichloromethane (100 ml) was added (solution A). In a separate flask L-menthyl-5R-acetoxy-1,3-oxathiolane-2R-carboxylate (50 g, 0.151 mol, 1.0 molar equivalents), zirconium tetrachloride (17.6 g, 0.075 mole, 0.5 molar equivalents) and dichloromethane (500 ml) were mixed under nitrogen atmosphere (solution B). Solution A was added into solution B under stirring at 25-30° C. and continued the stirring at the same temperature for 6 hrs. Reaction was monitored by HPLC or thin layer chromatography. After completion of reaction, the reaction mixture was cooled to 10-20° C. and MeOH (150 ml) was added under stirring. The mixture was concentrated under vacuum and stripped with 50 ml of MeOH at 40-50° C. Fresh MeOH (150-200 ml) was added and cooled the mixture to RT. The isolated solid was further cooled to 5-10° C. and maintained for an hour. The solid product was filtered and washed with pre-cooled (5-10° C.) MeOH. The solid product was dried under vacuum at 40-45° C. Yield: 45 g. Chiral Purity: 97% and 3% undesired isomer.

### Example 7

### Preparation of L-menthyl-5S-(4-amino-2-oxopyrimidin-1-(2H)-yl)-1,3-oxathiolane-2R-carboxylate

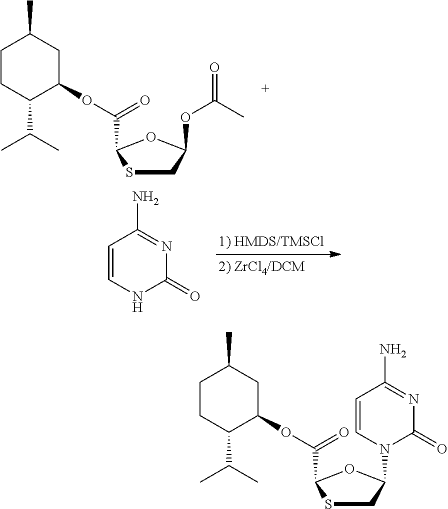

Cytosine (20 g, 0.180 mol, 1.2 molar equivalents), Hexamethyl disilazane (HMDS) (70 ml, 0.333 mol, 2.2 molar equivalents) and trimethylsilyl chloride (TMSCL) (10 ml) were mixed at 25-30° C. under Nitrogen atmosphere. The reaction mixture was heated up to 120-130° C. to get clear solution and excess solvent was distilled out under vacuum to get the residue of silylated cytosine. Fresh dichloromethane (100 ml) was added into silylated cytosine (solution A). In a separate flask L-menthyl-5R-acetoxy-1,3-oxathiolane-2R-carboxylate (50 g, 0.151 mol, 1.0 molar equivalents), dichloromethane (500 ml) and zirconium tetrachloride (ZrCl₄) (17.6 g, 0.075 mole, 0.5 molar equivalents) were mixed under nitrogen atmosphere (solution B). Presilylated cytosine solution A was added into solution B at 25-30° C. and reaction mixture was stirred at the same temperature for 6 hrs. The reaction was monitored by HPLC or thin layer chromatography. After completion of reaction, the reaction mixture was cooled to 10-20° C. and saturated sodium bicarbonate solution (250 ml) was added under stirring. The layers were separated and the organic layer was concentrated under vacuum to get the residue. To the residue isopropyl acetate (15-20 ml) was added under stirring. The mixture was cooled to 5-10° C. and maintained for 2-3 hours. The isolated solid was filtered and washed with isopropyl acetate. The solid was dried under vacuum at 40-45° C. Yield: 45 g.

### Example 8

### Preparation of L-menthyl-5S-(4-amino-5-fluoro-2-oxopyrimidin-1-(2H)-yl)-1,3-oxathiolane-2R-carboxylate

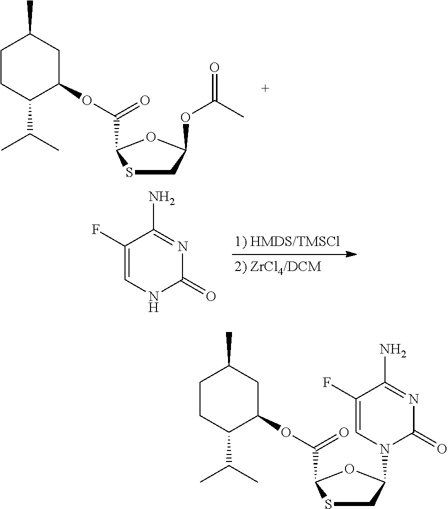

Fluorocytosine (22.4 g, 0.173 mol, 1.1 molar equivalents), Hexamethyl disilazane (HMDS) (100 ml, 0.477 mol, 3.1 molar equivalents) and trimethylsilyl chloride (TMSCL), (11.2 ml) were mixed at 25-30° C. under Nitrogen atmosphere. The reaction mixture was heated up to 120-130° C. to get clear solution and excess solvent was distilled out under vacuum at 90-100° C. to get the residue of silylated fluorocytosine. The residue of silylated fluorocytosine was cooled to room temperature and fresh dichloromethane (100 ml) was added (solution A). In a separate flask L-menthyl-5R-acetoxy-1,3-oxathiolane-2R-carboxylate (50 g, 0.151 mol, 1.0 molar equivalents), dichloromethane (250 ml) and zirconium tetrachloride (ZrCl₄) (17.6 g, 0.075 mole, 0.5 molar equivalents) were mixed under nitrogen atmosphere (solution B). Presilylated fluorocytosine solution A was added into solution B at 25-30° C. and reaction mixture was stirred at the same temperature for 6 hrs. The reaction was monitored by HPLC or thin layer chromatography. After completion of reaction, the reaction mixture was cooled to 10-20° C. and precooled (10-20° C.) water (250 ml) was added under stirring. pH was adjusted at 8 to 8.5 using triethylamine under stirring. The layers were separated and the organic layer was washed with 250 ml of water. The organic layer was concentrated under vacuum to get the residue. The residue was dissolved in methanol (200 ml) and n-heptane (100 ml) under stirring. The said solution was added into water (200 ml). The isolated solid was filtered and washed with water followed by n-heptane. The solid was dried under vacuum at 40-45° C. Yield: 48.5 g (80%). Chiral Purity: 99.94%; 0.06% undesired isomer. ¹H NMR (DMSO-d6) δ (ppm): 0.70-0.99 (m, 10H), 1.02-1.10 (m, 2H), 1.39-1.49 δ (m, 2H), 1.63-1.66 δ (d, 2H), 1.88-1.95 δ (m, 2H), 3.20-3.24 δ (m, 1H), 3.53-3.57 δ (m, 1H), 4.66-4.72 (m, 1H), 5.71 δ (s, 1H), 6.29-6.30 6 (d, 1H), 7.69 (s, 1H), 7.94 6 (s, 1H), 8.16-8.18 6 (d, 1H); ¹³C NMR (DMSO-d6): 16.5, 20.9, 22.2.3.2, 26.1, 31.8, 34, 35.8, 46.8, 76, 78.3, 89.5, 125.4, 135.2, 137.6, 153.4, 158, 169.8; IR (KBr) (cm⁻¹): 3323, 3083, 2956, 2869, 1754, 1687, 1640, 1513, 1348, 1287, 1178, 1090, 940, 774, 678, 498; MS (EI) m/z=400 (M+1); [α]_{D}²⁵=−45.23° (c=0.2%, MeOH).

### Example 9

### Preparation of 2-benzoyloxymethyl-5-(4-amino-2-oxopyrimidin-1-(2H)-yl)-1,3-oxathiolane

Cytosine (2 g, 0.018 mol, 1.1 molar equivalents), N,O-bis-(trimethylsilyl)-acetamide (BSA) (10 ml, 0.04 mol, 2.3 molar equivalents) and acetonitrile (10 ml) were mixed under nitrogen atmosphere at 25-30° C. to get clear solution. The solution was distilled under vacuum at 50-55° C. to remove excess BSA and acetonitrile completely to get the residue of silylated cytosine into which the dichloromethane (50 ml) was added (solution A). In a separate flask 2-benzoyloxymethyl-5-acetoxy-1,3-oxathiolane (40% cis isomer and 60% of trans isomer) (5 g, 0.017 mole, 1.0 molar equivalents), zirconium tetrachloride (1.76 g, 0.005 mol, 0.4 molar equivalents) and dichloromethane (50 ml) were mixed under nitrogen atmosphere (solution B). Solution A was added into solution B under stirring at 25-30° C. and continued the stirring at the same temperature for 6 hrs. Reaction was monitored by HPLC or thin layer chromatography. After completion of reaction, the reaction mixture was cooled to 10-20° C. in which saturated bicarbonate solution (50 ml) was added under stirring. The layers were separated and the organic layer was concentrated under vacuum to get the residue. The residual product was analyzed by HPLC which shows 40% of trans isomer and 60% of cis isomer.

### Example 10

### Preparation of 4-amino-1-[(2R,5S)-2-(hydroxymethyl)-1,3-oxathiolan-5-yl]-1,2-dihydropyrimidin-2-one (Lamivudine)

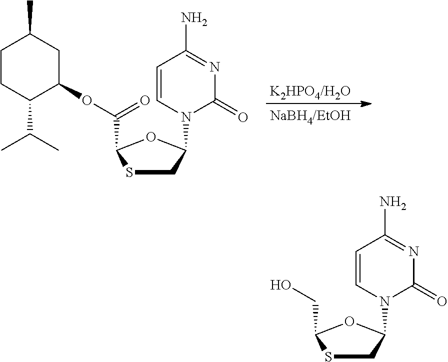

Di-potassium hydrogen phosphate (68.5 gm, 0.393 mol, 7.0 molar equivalents) and DM water (75 ml) was mixed at RT and L-menthyl-5S-(4-amino-2-oxopyrimidin-1-(2H)-yl)-1,3-oxathiolane-2R-carboxylate (50 g, 0.131 mol, 1.0 molar equivalents) in ethanol (375 ml) was added to it. The reaction mixture was cooled to 15-20° C. (solution A). In another flask sodium borohydride (NaBH₄) (10 g, 0.264 mol, 2.0 molar equivalents) in DM water (100 ml) containing 25% w/w NaOH was dissolved at 10-15° C. (solution B). Solution B was added into solution A at 15-20° C. The reaction was maintained at 15-20° C. and monitored by TLC or HPLC. After completion of reaction the layers were separated and adjusted the pH of upper layer to 4-4.5 using the concentrated HCl (12 ml) and then to pH 6.8-7.2 using 2M NaOH solution. The mixture was stirred and concentrated under vacuum. Water (150 ml) was added to the mixture followed by activated Carbon (5 gm). The mixture was heated up to 70-75° C. and maintained for 30 min. The mixture was filtered through Celite bed. The filtrate was cooled to RT and washed with toluene. Aqueous layer was concentrated under vacuum and co-distill the traces of water with methanol (50 ml×2). The residue was dissolved in methanol (300 ml) and heated to 60-65° C. for 30 min under stirring. The inorganic solid mixture was filtered and the filtrate was distilled under vacuum up to 1 volume remains in the flask. The mixture was stirred and isolated solid was filtered. The wet cake of solid product was washed with methanol (10 ml) and suck dried. The solid product was further dried under vacuum to afford crude Lamivudine. Yield: 25 g. Chiral Purity: cis-(−) Lamivudine=98% and cis-(+) Lamivudine=2%. ¹H NMR (MeOD) δ (ppm): 3.11-3.15 (m, 1H), 3.49-3.54 (m, 1H), 3.86-3.97 (m, 2H), 5.28 (t, 1H), 5.88-5.90 (d, 1H), 6.29 (t, 1H), 8.06-5.08 (d, 1H); ¹³C NMR (MeOD) δ (ppm): 38.5, 64, 88, 88.8, 95.7, 142.7, 157.9, 167.7; IR (KBr) (cm⁻¹): 3553, 3368, 3236, 1162, 1613, 1493, 1404, 1356, 1290, 1198, 1108, 1053, 967, 786, 696, 605, 539; MS (EI) m/z=229.9 (M+1); [α]_{D}²⁵=−98° (c=0.5%, water).

### Example 11

### Preparation of 4-amino-5-fluoro-1-[(2R,5S)-2-(hydroxymethyl)-1,3-oxathiolan-5-yl]-1,2-dihydropyrimidin-2-one (Emtricitabine)

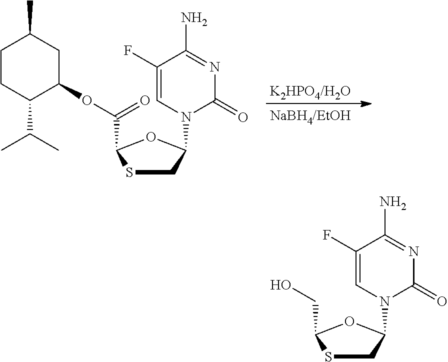

Di-potassium hydrogen phosphate (65.5 g, 0.376 mol, 3.0 molar equivalents) and DM water (75 ml) as mixed at RT and L-menthyl-5S-(4-amino-5-fluoro-2-oxopyrimidin-1-(2H)-yl)-1,3-oxathiolane-2R-carboxylate (50 g, 0.125 mol, 1.0 molar equivalents) in ethanol (350 ml) was added to it. The reaction mixture was cooled to 15-20° C. (solution A). In another flask sodium borohydride (NaBH₄) (13.5 g, 0.330 mol, 2.85 molar equivalents) in DM water (135 ml) containing 25% w/w NaOH was dissolved at 10-15° C. (solution B). Solution B was added into solution A at 15-20° C. The reaction was maintained at 15-20° C. and monitored by TLC or HPLC. After completion of reaction the layers were separated and adjusted the pH of upper layer to 4-4.5 using the concentrated HCl (20 ml) and then to pH 6.8-7.2 using 2M NaOH solution. The mixture was stirred and concentrated under vacuum. Water (150 ml) was added to the mixture followed by activated Carbon (5 gm). The mixture was heated up to 70-75° C. and maintained for 30 min. The mixture was filtered through Celite bed. The filtrate was cooled to RT and washed with toluene. Aqueous layer was concentrated under vacuum and co-distill the traces of water with methanol (50 ml×2). The residue was dissolved in methanol (300 ml) and heated to 60-65° C. for 30 min under stirring. The inorganic solid mixture was filtered and the filtrate was distilled under vacuum up to 1 volume remains in the flask. The mixture was stirred and isolated solid was filtered. The wet cake of solid product was washed with methanol (10 ml) and suck dried. The solid product was further dried under vacuum to afford crude Emtricitabine. Yield: 25 g. Chiral Purity: cis-(−) Emtricitabine=98% and cis-(+) Emtricitabine=2%. ¹H NMR (DMSO-d6) δ (ppm): 3.10-3.14 (m, 1H), 3.40-3.44 (m, 1H), 3.70-3.81 (m, 2H), 5.17-5.19 (t, 1H), 5.43-5.45 (t, 1H), 6.13-6.15 (t, 1H), 7.59 6 (s, 1H), 7.84 6 (s, 1H), 8.20-8.22 (d, 1H); ¹³C NMR (DMSO-d6) δ (ppm): 37.2, 62.5, 87, 126.2, 135, 137.4, 153.4, 158; IR (KBr) (cm⁻¹): 3420, 3248, 3104, 3083, 29.02, 1695, 1625, 1520, 1407, 1343, 1298, 1251, 1169, 1092, 776, 619, 465; MS (EI) m/z=246 (M−1); [α]_{D}²⁰=−106.47° (c=0.25%, water), [α]_{D}²⁵=−141.37° (c=1%, MeOH).

### Example 12

### Preparation of (−)-1-(2R,Cis)-4-amino-1-[(2-hydroxymethyl)-1,3-oxathiolan-5-yl]-2(1H)-pyrimidin-2-one-(S)-Binol Co-Crystal

S-(−)-BINOL (50 g) and methanol (200 ml) was mixed at 30-35° C. to get a clear solution. Crude Lamivudine (25 gm) was added into the solution and heated the mixture up to 60-65° C. to get clear solution. The solution was cooled to ambient temperature at rate of 10° C./hr and stirred for 2-4.5 hrs. The isolated solid was filtered and washed with methanol (25 ml). The solid product was suck dried and further dried under vacuum to get the S-(−)-BINOL Co-crystal. Yield: 47 g. Chiral Purity: cis (−) Lamivudine=99.25% and cis (+) Lamivudine=0.75%.

### Example 13

### Preparation of 4-amino-1-[(2R,5S)-2-(hydroxymethyl)-1,3-oxathiolan-5-yl]-1,2-dihydropyrimidin-2-one (Lamivudine)

Lamivudine (S)-BINOL Co-crystal (20 g) in ethyl acetate (100 ml) and D.M. water (200 ml) were mixed at RT to obtain a clear solution which was then heated up to 35-40° C. and maintained for 15 min. The solution was cooled to RT and the layers were separated. Aqueous layer was washed with Ethyl acetate (100 ml) and both the ethyl acetate layers were combined and extracted with water (100 ml). Both the aqueous layer was combined and charcolized with activated carbon (2.0 gm) at 45-50° C. under stirring. The charcolized mixture was filtered through celite bed and washed the celite bed with 20 ml chloride free water. The filtrate was distilled out under vacuum at 45-50° C. till final volume becomes 1.2 volumes. Denatured spirit (DNS) (8 ml) was added to the mixture and was heated to 45-50° C. The mixture was filtered through 0.5μ. The filtrate was cooled to 30-32° C. and seeded with 0.025 gm of Lamivudine Form-1. The mixture was rapidly cooled to 8-10° C. and maintained the isolated solid under stirring for 1 hr. The isolated solid was filtered and washed the wet cake with 4 ml of pre-cooled (8-10° C.) DM Water and DNS mixture (3:1). The solid was suck dried and dried under vacuum to afford Lamivudine. Yield: 7 g. Chiral Purity: cis-(−) Lamivudine=99.8%.

### Example 14

### Preparation of 4-amino-1-[(2R,5S)-2-(hydroxymethyl)-1,3-oxathiolan-5-yl]-1,2-dihydropyrimidin-2-one (Lamivudine)

(S) BINOL Co-crystal (20 g), ethyl acetate (100 ml) and D.M. water (100 ml) were mixed at RT. Concentrated HCl (4 ml) was added to adjust the pH 3-4 and the mixture was stirred for 5 min. The layers were separated and the aqueous layer was washed with fresh ethyl acetate (100 ml). The pH of aqueous layer was adjusted to 6.8-7.2 using 10% NaOH (10 ml). The aqueous layer was passed through activated resin 225-H column and the column was washed with chloride free water. 10% aqueous ammonia solution was added to resin column to elute the product. After the completion of elution, distilled out the solvent till 10 ml remains in the flask. Fresh chloride free water (100 ml) and activated carbon (2.0 gm) was added and heated the mixture up to 45-50° C. The mixture was stirred and filtered through celite bed which was washed with 20 ml chloride free water. The solution was distilled under vacuum at 45-50° C. till 1.2 volumes remains in the flask. DNS (8 ml) was added to the solution and stirred for 5 min. The solution was filtered through 0.5μ and cooled to 30-32° C. The solution was seeded with of 0.025 g Lamivudine Form-I at 30-32° C. The mixture was further cooled up to 8-10° C. and stirred the isolated solid for 1 hrs. The solid was filtered and washed the wet cake with 4 ml of pre-cooled (8-10° C.) DM Water and DNS mixture (3:1). The solid product was suck dried and further dried under vacuum to afford Lamivudine. Yield: 7 g. Chiral Purity: cis-(−) Lamivudine=99.8%.

This invention has been described with reference to its preferred embodiments. Variations and modifications of the invention will be obvious to those skilled in the art from the foregoing detailed description of the invention. It is intended that all of these variations and modifications be included within the scope of this invention.

1. A process for the preparation of 1,3-oxathiolane nucleosides and stereoisomers thereof having the general formula (I) 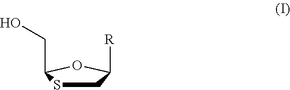
wherein R is substituted or unsubstituted purine or pyrimidine base,
comprising a step of stereoselective glycosylation wherein a compound of formula (V) 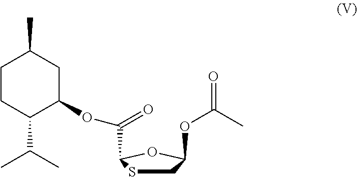
is reacted with optionally silylated purine or pyrimidine base in presence of ZrCl₄ in an organic solvent at a temperature ranging between 20-40° C.

2. The process according to claim 1, wherein the pyrimidine base is selected from cytosine or fluorocytosine.

3. The process according to claim 1, wherein the optionally silylated cytosine or 5-fluorocytosine is optionally acylated at amino function.

4. The process according to claim 1, wherein 1,3-oxathiolane nucleosides is Lamivudine.

5. The process according to claim 1, wherein 1,3-oxathiolane nucleosides is Emtricitabine.

6. The process according to claim 1, wherein the organic solvent is dichloromethane.

7. The process according to claim 1, wherein the mole of zirconium chloride (ZrCl₄) with respect of compound (V) is 0.5 mol.

8. The process as claimed in claim 1, wherein compound obtained after the reaction of compound (V) with optionally silylated purine or pyrimidine base is further reduced in presence of metal hydride.

9. The process as claimed in claim 8, wherein the metal hydride is sodium borohydride.
